(19) United States
(12) Patent Application Publication
(10) Pub. No.: US 2026/0175028 A1
(43) Pub. Date: Jun. 25, 2026

(54) TREATMENTS FOR NEURAL DYSREGULATION

(71) Applicant: SynchNeuro, Inc., Philadelphia, PA (US)

(72) Inventor: Emily MIRRO, Pacifica, CA (US)

(21) Appl. No.: 19/127,073

(22) PCT Filed: Nov. 3, 2023
(86) PCT No.: PCT/US2023/078710

§ 371 Date: May 2, 2025

Related U.S. Application Data
(60) Provisional application No. 63/382,235, filed on Nov. 3, 2022.

(51) Int. Cl. A61N 1/36; A61N 1/20; A61N 2/00

(52) U.S. Cl. CPC A61N 1/36085; A61N 1/20; A61N 1/36132; A61N 1/36139; A61N 2/006

(57) ABSTRACT

Methods, devices and systems for treating dysregulated eating behavior in a subject, in a subject with an impaired orexigenic appetitive processing circuit mediating activity between a lateral hypothalamus (LH) and a dorsolateral hippocampus (dlHPC), including invasively and/or non-invasively delivering neurostimulation to at least a portion of the impaired subject to at least partially repair the impaired circuit.

### CROSS REFERENCE TO RELATED APPLICATIONS

This application claims the priority of U.S. Provisional Application No. 63/382,235, filed Nov. 3, 2022, which is incorporated by reference herein in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

The disclosures of U.S. Pat. Nos. 8,909,342 and 8,725,244 are incorporated by reference herein in their entireties for all purposes.

### BACKGROUND

Barbosa DAN, Gattas S, Salgado J S, et al. A hedonic orexigenic subnetwork within the human hippocampus. Research Square; 2022. DOI: 10.21203/rs.3.rs-1315996/v1 (“Barbosa”) summarizes a structural/functional orexigenic appetitive processing circuit mediating activity between the lateral hypothalamus (LH) and dorsolateral hippocampus (dlHPC) in humans. As reported, this circuit is intact in people with normal eating behavior, but is impaired in people with dysregulated eating behavior. An impaired circuit mediating activity between the lateral hypothalamus (LH) and dorsolateral hippocampus (dlHPC) may be referred to herein as “the circuit.”

FIG. 7 illustrates four figures showing brain section images from “Barbosa”, and labels dorsolateral hippocampus (dlHPC) 102 and 102′ (contrasted with non-dlHPC) and lateral hypothalamus (LH) 104 and 104′.

Neurostimulation modulates the depolarization of neurons which results in neuronal plasticity and repair. Hogan M K, Hamilton G F, Horner P J. Neural Stimulation and Molecular Mechanisms of Plasticity and Regeneration: A Review. Front Cell Neurosci. 2020 Oct. 14; 14:271. doi: 10.3389/fncel.2020.00271. PMID: 33173465; PMCID: PMC7591397. Literature supports that forms of invasive and non-invasive neurostimulation repair dysfunctional brain circuits in stroke, traumatic brain injury, and nerve damage (Ting W K, Fadul F A, Fecteau S, Ethier C. Neurostimulation for Stroke Rehabilitation. Front Neurosci. 2021 May 14; 15:649459. doi: 10.3389/fnins.2021.649459; Veldema, J., Gharabaghi, A. Non-invasive brain stimulation for improving gait, balance, and lower limbs motor function in stroke. J NeuroEngineering Rehabil 19, 84 (2022). doi: 10.1186/s12984-022-01062-y; Bender Pape, Theresa L. DrPH, MA, CCC-SLP/L, FACRM; Editor; Herrold, Amy A. PhD; Guernon, Ann PhD, MS, CCC-SLP/L; Aaronson, Alexandra MD; Rosenow, Joshua M. MD, FACS; Co-editors. Neuromodulatory Interventions for Traumatic Brain Injury. Journal of Head Trauma Rehabilitation: November/December 2020; and Meyers, E. C., Kasliwal, N., Solorzano, B. R. et al. Enhancing plasticity in central networks improves motor and sensory recovery after nerve damage. Nat Commun 10, 5782 (2019). doi: 10.1038/s41467-019-13695-0).

Approaches are needed that can repair the circuit in subjects with dysregulated eating behavior.

### SUMMARY OF THE DISCLOSURE

This disclosure is related to methods, devices, and systems for repairing an impaired circuit mediating activity between the lateral hypothalamus (LH) and dorsolateral hippocampus (dlHPC) with one or more form of invasive or non-invasive neurostimulation that will repair the circuit neuroplasticity, and resolve or at least partially treat the dysregulated eating behavior.

One aspect of the disclosure is a method of treating dysregulated eating behavior in a subject, comprising: in a subject with an impaired orexigenic appetitive processing circuit mediating activity between a lateral hypothalamus (LH) and a dorsolateral hippocampus (dlHPC), invasively and/or non-invasively delivering targeted neurostimulation to at least a portion of the impaired circuit to at least partially repair the impaired circuit.

In this aspect, invasively and/or non-invasively delivering neurostimulation to at least a portion of the impaired circuit may comprise invasively and/or non-invasively delivering targeted neurostimulation to at least one of the LH or the dlHPC.

In this aspect, the method may include invasively delivering targeted neurostimulation to at least partially repair the impaired circuit.

In this aspect, invasive neurostimulation may comprise invasively delivering neurostimulation to at least one of the LH or the dlHPC to at least partially repair the impaired circuit.

In this aspect, invasive neurostimulation may comprise continuous deep brain stimulation (“DBS”) to at least one of the LH or the dlHPC.

In this aspect, invasive neurostimulation may comprise closed-loop DBS to at least one of the LH or the dlHPC. Closed-loop DBS may be responsive to low frequency (4-6 Hz) in the dlHPC known to modulate sweet-fat cue.

In this aspect, invasive neurostimulation may comprise scheduled DBS to at least one of the LH or the dlHPC. Scheduled DBS may occur during at least one of only during the day, only during the night, or only at mealtimes.

In this aspect, invasive neurostimulation may comprise at least partially patient-controlled DBS to at least one of the LH or the dlHPC, optionally wherein the patient initiates stimulation using an external patient controller and/or a patient magnet. Patient-controlled DBS may occur at or near times of craving.

In this aspect, invasive neurostimulation may comprise continuous or duty-cycle delivery of stimulation to a vagus nerve.

In this aspect, invasive neurostimulation may comprise patient-controlled delivery of stimulation to a vagus nerve.

In this aspect, the method may include non-invasively delivering targeted neurostimulation to at least partially repair the impaired circuit. Non-invasively delivering targeted brain neurostimulation may comprise repetitive transcranial magnetic stimulation (rTMS) delivery. Non-invasively delivering targeted brain neurostimulation may comprise continuous transcranial magnetic stimulation (cTMS) delivery. Non-invasively delivering targeted brain neurostimulation may comprise transcranial direct current stimulation (tDCS) delivery. Non-invasively delivering targeted brain neurostimulation may comprise transcranial alternating stimulation (tACS) delivery.

In this aspect, delivering neurostimulation may comprise delivering high frequency stimulation (>100 Hz).

In this aspect, delivering neurostimulation may comprise delivering low frequency stimulation (<4 Hz).

In this aspect, delivering neurostimulation may comprise low frequency stimulation matching the low frequency EEG signal power described in the Barbosa article incorporated by reference (4-6 Hz).

In this aspect, delivering neurostimulation may comprise stimulating the impaired circuit.

This aspect may include any one or more features described and/or shown in this application.

One aspect of this disclosure is a method of treating dysregulated eating behavior in a subject, comprising: in a subject with an impaired orexigenic appetitive processing circuit mediating activity between a lateral hypothalamus (LH) and a dorsolateral hippocampus (dlHPC), invasively and/or non-invasively delivering brain neurostimulation to at least one of the LH or dlHPC to at least partially repair the impaired circuit.

One aspect of the disclosure is a system, comprising: one or more processors; and one or more storage media coupled to the one or more processors and storing instructions that, when executed by the one or more processors, performs a computer-implemented method comprising: causing neurostimulation to be delivered to at least a portion of impaired orexigenic appetitive processing circuit mediating activity between a lateral hypothalamus (LH) and a dorsolateral hippocampus (dlHPC).

In this aspect, the one or more processors and the one or more storage media are optionally disposed in an implantable medical device (e.g., in component 18 in FIG. 1) or optionally in an external device (e.g., for non-invasive stimulation), including any of the non-invasive devices and systems herein.

In this aspect, the storing instructions that, when executed by the one or more processors, perform a computer-implemented method that may comprise causing continuous DBS to be delivered to at least a portion of the impaired circuit.

In this aspect, the storing instructions that, when executed by the one or more processors, perform a computer-implemented method that may comprise closed-loop DBS to at least a portion of the impaired circuit. Closed-loop DBS may be responsive to sensed low frequency (4-6 Hz) in the dlHPC known to modulate sweet-fat cue.

In this aspect, the storing instructions that, when executed by the one or more processors, perform a computer-implemented method may comprise scheduled DBS to at least a portion of the impaired circuit. Scheduled DBS may be programmed to occur during at least one of only during the day, only during the night, or only at mealtimes.

In this aspect, the storing instructions that, when executed by the one or more processors, perform a computer-implemented method may comprise at least partially patient-controlled DBS to at least a portion of the impaired circuit. The system may optionally comprise an implantable pulse generator, and wherein the implantable pulse generator may be adapted to be communication with a patient controller, wherein the patient controller, in response to user actuation of the patient controller, may communicate with the implantable pulse generator and facilitate the initiation of the DBS.

In this aspect, the system may be a non-invasive neurostimulation system. Non-invasive systems may comprise a repetitive transcranial magnetic stimulation device, a continuous transcranial magnetic stimulation device, a transcranial direct current stimulation device, or a transcranial alternating stimulation device, or optionally some combination thereof.

In this aspect, neurostimulation may comprise delivering high frequency stimulation (>100 Hz) to at least a portion of the impaired circuit.

In this aspect, neurostimulation may comprise delivering low frequency stimulation (<4 Hz) to at least a portion of the circuit.

In this aspect, neurostimulation may comprise low frequency stimulation matching the low frequency EEG signal power (4-6 Hz) described in the Barbosa article incorporated by reference.

In this aspect, the system may further include one or more intracranial leads, each including one or more electrodes thereon.

### BRIEF DESCRIPTION OF THE DRAWINGS

FIG. 1 is a conceptual diagram illustrating at least a portion of an exemplary system that includes an implantable medical device (which may be considered an implantable pulse generator) implanted in the chest and once or more leads, and that is adapted for deep brain stimulation (DBS).

FIG. 2 is a conceptual diagram illustrating another example system that includes an implantable medical device implanted under the scalp and once or more leads, and that is adapted for deep brain stimulation.

FIG. 3 is a block diagram illustrating exemplary systems and implantable medical devices of FIGS. 1 and 2.

FIG. 4 is a conceptual diagram illustrating another exemplary system that includes one or more of a clinician device and a patient control device (patient controller).

FIG. 5 illustrates at least a portion of an exemplary method of at least partially repairing an impaired circuit.

FIG. 6 illustrates an exemplary method of at least partially treating the circuit that includes closed loop DBS.

FIG. 7 illustrates brain regions of interest (dlHPC and LH), wherein neurostimulation of the impaired circuit, optionally stimulation of at least one of the brain regions of interest, can at least partially repair a dysregulated LH-dlHPC circuit to treat a dysregulated eating behavior.

### DETAILED DESCRIPTION

This disclosure is related to methods of repairing dysregulated LH-dlHPC circuits, the methods optionally comprising invasive and/or non-invasive neurostimulation adapted to repair the circuit neuroplasticity, and thereby at least partially treat the dysregulated eating behavior (e.g., eating disorders). The methods herein generally at least partially repair the impaired neural connectivity between an LH and a dlHPC in a subject with a dysregulated eating behavior.

One aspect of the disclosure is a method of treating dysregulated eating behavior (e.g., an eating disorder) in a subject with an impaired orexigenic appetitive processing circuit mediating activity between a lateral hypothalamus (LH) and a dorsolateral hippocampus (dlHPC). The method may comprise repairing the impaired orexigenic appetitive processing circuit mediating activity between the lateral hypothalamus (LH) and the dorsolateral hippocampus (dlHPC).

Treatments herein that at least partially repair the circuit may comprise invasive and/or non-invasive neurostimulation to at least a portion of the circuit.

Examples herein that deliver invasive neurostimulation may comprise deep brain stimulation (“DBS”), general concepts of which are known and are incorporated by reference herein. For example, DBS may include one or more leads implanted in the brain at one or more particular locations, the one or more leads including one or more electrodes thereon adapted for sensing electrical brain activity and/or delivering neurostimulation to the brain.

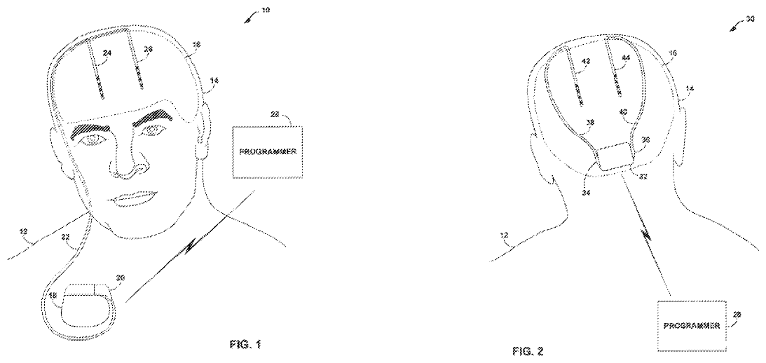

FIG. 1 is a conceptual diagram illustrating an example system 10 that includes an implantable medical device (IMD) 18, which may also be referred to herein as an implantable pulse generator (IPG) implanted in the chest of a patient 12. IMD 18 may be adapted to one or both of receive electrical information sensed by the lead electrodes or generate signals to the lead electrodes to thereby apply the DBS. As is described in more detail below, system 10 may be adapted for and used with continuous DBS (with or without sensing); closed-loop (intermittent) DBS, which may initiate DBS in response to detection/sensing of one or more types of brain signals (e.g., 4-6 Hz); scheduled or programmed DBS (e.g., during one or more programmed epochs of time, such as only at night, only during the day, during mealtimes, etc.); and/or patient controlled DBS in which the subject initiates the DBS using a patient controller or patient magnet.

In some closed-loop embodiments, IMD 18 may be adapted to automatically process sensed brain activity information and autonomously and automatically modify the DBS therapy.

In the illustrated example, IMD 18 takes the form of an implantable neurostimulator that delivers neurostimulation therapy in the form of electrical pulses to patient 12 via leads 24 and 26. However, the IMD may be positioned at other locations.

In the example of FIG. 1, IMD 18 delivers neurostimulation therapy to patient 12 via leads 24 and 26, which are connected to IMD 18 via a lead extension 22. Lead extension 22 couples to IMD 18 via connector 20. Leads 24 and 26 may, as shown in FIG. 1, be implanted within the brain of patient 12, and IMD 18 may deliver stimulation therapy to the brain, e.g., deep brain stimulation (DBS). In the illustrated example, leads 24 and 26 may be implanted at similar locations in each the right and left hemisphere of brain 16. In this manner, IMD 18 may deliver stimulation to bilateral locations within brain 16.

Leads may be positioned at or proximate the LH and/or the dlHPC such that the DBS stimulates at least a portion of the impaired circuit, repairs its neuroplasticity, and thereby at least partially treats the dysregulated eating behavior.

However, the disclosure herein is not limited to the configuration of leads 24 and 26 or extensions 22 shown in FIG. 1. In other embodiments, non-symmetrical leads or a single lead may be used to deliver DBS therapy. In other words, one or more leads 24 and 26 may be coupled directly to IMD 18, or be coupled to IMD 18 by one or more extensions 22, and may extend from IMD 18 to any one or more portions of brain 16.

Additionally or alternatively, one or more of leads 24 and 26 may be implanted proximate to a vagal nerve to provide DBS to the vagal nerve to repair the circuit. In alternative embodiments, the vagal nerve at or near the neck region may have an electrode cuff disposed about it, as is common in some vagal nerve stimulation systems, such as those that are adapted to treat epilepsy with vagal nerve stimulation with a nerve cuff with one or more electrodes, and an IPG.

IMD 18 may deliver either or both of responsive, e.g., closed-loop, or non-responsive stimulation (e.g., continuous). An example of responsive stimulation is delivery of DBS in response to detection of electrical activity within the brain of patient 12 associated with a sweet-fat cue, for example.

IMD 18 delivers therapy according to a set of therapy parameters, i.e., a set of values for a number of parameters that define the therapy delivered according to that therapy parameter set. In embodiments where IMD 18 delivers neurostimulation therapy in the form of electrical pulses, the parameters in each parameter set may include voltage or current pulse amplitudes, pulse widths, pulse rates, and the like. Further, each of leads 24 and 26 may include one or more electrodes disposed at the distal end or distal region of each lead, and a therapy parameter set may include information identifying which electrodes have been selected for delivery of pulses, and the polarities of the selected electrodes. Therapy parameter sets used by IMD 18 may include parameter sets programmed by a clinician (not shown), and parameter sets representing adjustments made by patient 12 to these preprogrammed sets. In some embodiments, adjustments or modifications to therapy parameter sets may be performed automatically or suggested to patient 12 by IMD 18 or other components of system 10, such as a programmer.

In the illustrated example, system 10 includes an optional programmer 28. Programmer 28 may be a clinician or patient programmer that communicates with IMD 18, and system 10 may include any number of programmers 28 which may act as clinician or patient programmers. A clinician (not shown) may use programmer 28 to program therapy for patient 12, e.g., specify a number of therapy parameter sets and communicate the parameter sets to IMD 18. The clinician may also use programmer 28 to retrieve information collected by IMD 18. The clinician may use programmer 28 to communicate with IMD 18 both during initial programming of IMD 18, and for collection of information and further programming during follow-up visits.

Programmer 28 may include a display (not shown) to present information to the user and an input mechanism (not shown), e.g., a keypad, that allows the user to interact with the programmer. In some embodiments, the display may be a touch screen display, and a user may interact with programmer 28 via the display. A user may also interact with clinician programmer 28 using peripheral pointing devices, such as a stylus or mouse. The keypad may take the form of an alphanumeric keypad or a reduced set of keys associated with particular functions.

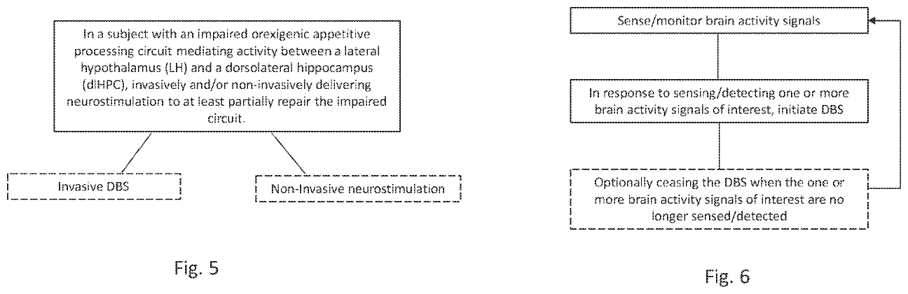

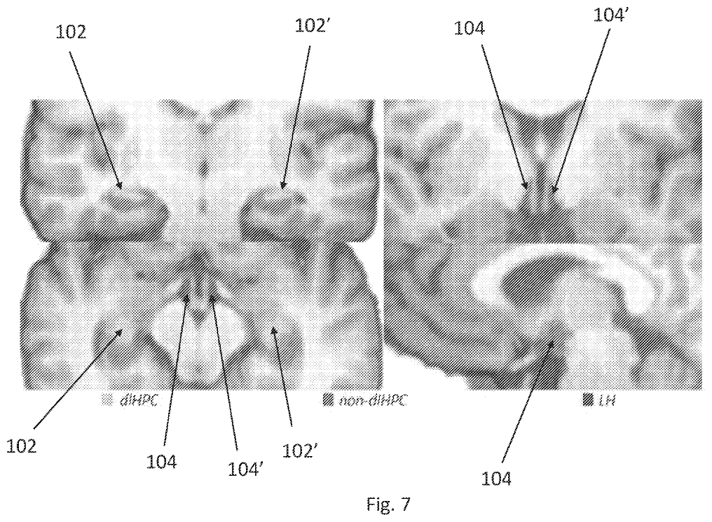

Programmer 28 may be embodied similar to clinician programmer 128 or patient programmer 134 (which may be used for patient-controlled and initiated DBS) of FIG. 5. However, programmer 28 is not limited to the embodiments depicted in FIG. 5.

As described above, programmer 28 may be a patient programmer. Patient 12 may use programmer 28 to control one or more aspects of the delivery of therapy by IMD 18 with systems that are adapted for patient-controlled DBS. For example, the subject may initiate the neurostimulation in response to one or more events, such as cravings. Additionally, and for example only, using programmer 28, patient 12 may select a current therapy parameter set from among the therapy parameter sets preprogrammed by the clinician, or may adjust one or more parameters of a preprogrammed therapy parameter set to arrive at the current therapy parameter set.

Programmer 28 may be any type of computing device. For example, programmer 28 may be a hand-held or tablet-based computing device, a desktop computing device, or a workstation. In addition, programmer 28 may be a virtual programmer in that a remote user may communicate with IMD 18 without being in the same room as patient 12.

IMD 18 and programmer 28 may communicate via wireless communication. Programmer 28 may communicate via wireless communication with IMD 18 using radio frequency (RF) telemetry techniques known in the art. Possible communications may follow RF protocols according to the 802.11 or Bluetooth specification sets, infrared communication according to the IRDA specification set, or other standard or proprietary telemetry protocols.

As mentioned above, IMD 18 may collect information relating to sensed brain electrical activity of the patient 12, such as with closed loop DBS systems. For example, IMD 18 may monitor brain electrical activity and/or electroencephalogram (EEG) morphology.

FIG. 2 is a conceptual diagram illustrating another example system 30 that includes an IMD 32. System 30 includes IMD 32, connection ports 34 and 36, leads 42 and 44 implanted within brain 16, and programmer 28. IMD 32 is substantially similar to IMD 18 of FIG. 1. However, IMD 32 is configured to be implanted beneath the scalp of head 14. In some embodiments, IMD 32 may be implanted at least partially within the skull of patient 12, e.g., within a recess or hole formed in or through the skull. Implanting IMD 32 in head 14 of patient 12 may reduce the length of leads 42 and 44 and reduce number of areas that must be surgically altered in the patient for implantation of the IMD. Implantation of an IMD in head 14, as illustrated in FIG. 2, is an alternative to implantation of an IMD within the chest of the patient, as illustrated in FIG. 1. However, the invention is not limited to the implantation locations illustrated in FIGS. 1 and 2. An IMD may be implanted anywhere within a patient. Leads 42 and 44 are tunneled from IMD 32 under the scalp of patient 12 to the location where each lead enters the skull of patient 12. Similar to leads 24 and 26 of FIG. 1, leads 42 and 44 may be symmetrical or stereotactic leads, i.e., both leads are implanted at similar locations in each the right and left hemisphere of brain 16. In this manner, IMD 32 may deliver stimulation to bilateral locations within brain 16. Other therapies may also be provided via leads 42 and 44 or other leads coupled to IMD 32. In some embodiments, connection ports 34 and 36 may be located at a different location on IMD 32 to provide alternative positions of leads 42 and 44. Any and all disclosure related to FIG. 1 is incorporated by reference into the embodiment shown in FIG. 2.

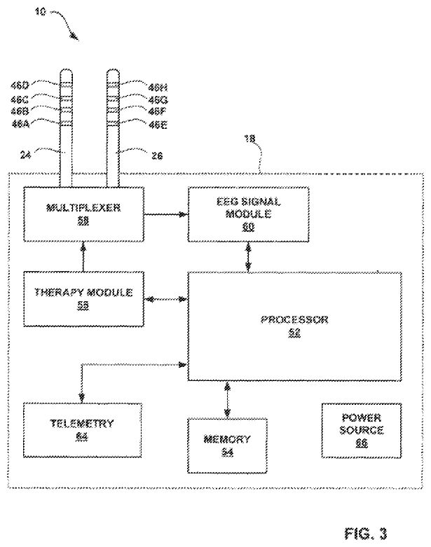

FIG. 3 is an illustrative and exemplary block diagram illustrating the example system and implantable medical device of FIGS. 1 and 2. IMD 18 of system 10 is shown as an example in FIG. 3; however, the block diagram may also be applicable to similar IMD 32 of system 30 or other systems herein. FIG. 3 illustrates an example configuration of IMD 18 and leads 24 and 26. It is understood that IMD 18 shown in FIG. 3 need not include all of the components shown, and may also include additional components not shown in FIG. 3.

IMD 18 may deliver DBS therapy via electrodes 46A-D of lead 24 and electrodes 46E-H of lead 26 (collectively “electrodes 46”). The leads may be more or fewer electrodes than shown. Electrodes 46 may be ring electrodes. The configuration, type and number of electrodes 46 illustrated in FIG. 3 are merely exemplary. For example, leads 24 and 26 may each include eight or any other number of electrodes 46, and the electrodes 46 need not be arranged linearly on each of leads 24 and 26 or be ring electrodes.

Electrodes 46 may be electrically coupled to an optional multiplexer 58. Multiplexer 58 is able to selectively couple each of the electrodes to circuits within IMD 18 under the control of a processor 52. For example, through multiplexer 58, processor 52 may selectively couple electrodes 46 to a therapy module 56 or EEG signal module 60.

Therapy module 56 may, for example, include an output pulse generator (PG) coupled to a power source 66, which may include a primary or rechargeable battery. Therapy module 56 may deliver electrical pulses to patient 12 via at least some of electrodes 46 under the control of a processor 52, which controls therapy delivery module 56 to deliver neurostimulation therapy according to a current therapy parameter set. The therapy parameter sets used by processor 52 to control delivery therapy by therapy module 56 may be received via a telemetry module 64 and/or stored in memory 54.

Processor 52 may include a microprocessor, a controller, a digital signal processor (DSP), an application specific integrated circuit (ASIC), a field-programmable gate array (FPGA), discrete logic circuitry, or the like. Memory 54 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, and the like. In some embodiments, memory 54 stores program instructions that, when executed by processor 52, cause IMD 18 and processor 52 to perform the functions attributed to them as described herein.

Optional EEG signal module 60 receives signals from a selected set of the electrodes 46 via multiplexer 58 as controlled by processor 52. EEG signal module 60 may analyze the EEG signal for certain features that are part of a closed loop detection system, the detection of which can trigger the DBS. IMD 18 may include circuitry (not shown) that conditions the EEG signal such that it may be analyzed by processor 52. For example, IMD 18 may include one or more analog to digital converters to convert analog signals generated by sensor 50 into digital signals usable processor 52, as well as suitable filter and amplifier circuitry.

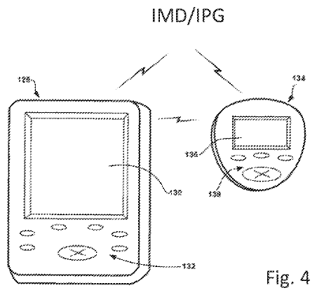

FIG. 4 also illustrates a system as including a clinician programmer 128 and a patient programmer 134. Clinician programmer 128 and patient programmer 134 may be similar to programmer 28 of FIGS. 1 and 2. A clinician (not shown) may use clinician programmer 128 to program therapy for patient 12, e.g., specify a number of therapy parameter sets and provide the parameter sets to IMD 122 (or IPG). The clinician may also use clinician programmer 128 to retrieve information collected by IMD 122. The clinician may use clinician programmer 128 to communicate with IMD 122 both during initial programming of IMD 122, and for collection of information and further programming during follow-up visits.

Clinician programmer 128 may, as shown in FIG. 4, be a handheld computing device. Clinician programmer 128 includes a display 130, such as an LCD or LED display, to display information to a user. Clinician programmer 128 may also include a keypad 132, which may be used by a user to interact with clinician programmer 128. In some embodiments, display 130 may be a touch screen display, and a user may interact with clinician programmer 128 via display 130. A user may also interact with clinician programmer 128 using peripheral pointing devices, such as a stylus or mouse. Keypad 132 may take the form of an alphanumeric keypad or a reduced set of keys associated with particular functions.

Patient programmer 134 also may, as shown in FIG. 4, be a handheld computing device. Patient 12 may use patient programmer 134 to control the delivery of therapy by IMD 122. For example only, the patient may initiate DBS in response to one or more patient events, such as cravings. Additionally for example, using patient programmer 134, patient 12 may select a current therapy parameter set from among the therapy parameter sets preprogrammed by the clinician, or may adjust one or more parameters of a preprogrammed therapy parameter set to arrive at the current therapy parameter set.

Patient programmer 134 may include a display 136 and a keypad 138, to allow patient 12 to interact with patient programmer 134. In some embodiments, display 136 may be a touch screen display, and patient 12 may interact with patient programmer 134 via display 136. Patient 12 may also interact with patient programmer 134 using peripheral pointing devices, such as a stylus, mouse, or the like.

However, clinician and patient programmers 128, 134 are not limited to the hand-held computer embodiments illustrated in FIG. 4. Programmers 128, 134 according to the invention may be any sort of computing device. For example, a programmer 128, 134 according to the invention may be a tablet-based computing device, a desktop computing device, or a workstation. IMD 122, clinician programmer 128 and patient programmer 134 may, as shown in FIG. 5, communicate via wireless communication. Clinician programmer 128 and patient programmer 134 may, for example, communicate via wireless communication with IMD 122 using radio frequency (RF) telemetry techniques known in the art. Clinician programmer 128 and patient programmer 134 may communicate with each other using any of a variety of local wireless communication techniques, such as RF communication according to the 802.11 or Bluetooth specification sets, infrared communication according to the IRDA specification set, or other standard or proprietary telemetry protocols.

Clinician programmer 128 and patient programmer 134 need not communicate wirelessly, however. For example, programmers 128 and 134 may communicate via a wired connection, such as via a serial communication cable, or via exchange of removable media, such as magnetic or optical disks, or memory cards or sticks. Further, clinician programmer 128 may communicate with one or both of IMD 122 and patient programmer 134 via remote telemetry techniques known in the art, communicating via a local area network (LAN), wide area network (WAN), public switched telephone network (PSTN), or cellular telephone network, for example.

Any of the disclosure described herein related to FIGS. 1-4 may be incorporated by reference into any of the other suitably combinable embodiments or examples herein, unless indicated herein to the contrary.

In some implementations that deliver invasive neurostimulation, the method and systems may include vagus nerve stimulation, which may optionally comprise coupling of nerve cuff around the vagus nerve in the vicinity of the neck, which is in operable communication with an implantable pulse generator, such as IMD 18 shown herein.

Any of the devices and systems herein (including in one or more of FIGS. 1-4) may be used with any of the treatments herein to repair the impaired circuit, which are described below.

In some implementations of the treatment to repair the impaired circuit, the neurostimulation may be delivered non-invasively, such as with one or more devices positioned on a subject's scalp.

For example only, non-invasive neurostimulation may comprise transcranial magnetic stimulation (TMS) delivery, such as repetitive transcranial magnetic stimulation (rTMS) delivery. Transcranial magnetic stimulation (TMS) is a non-invasive stimulation of brain tissue through the production of the high or low-intensity magnetic field to modulate cortical excitability. Repetitive transcranial magnetic stimulation (rTMS) refers to applying recurring TMS pulses to a specific brain region, which herein may be applied to at least a portion of the circuit, such as the LH and/or the dlHPC to repair the circuit. Generlly, rTMS has been classified as high frequency (>1 Hz), which increases the cortical excitability, and low frequency (<1 Hz), which depresses the cortical excitability. The rTMS is a brain stimulation technique in which the patient is seated with a large wire coil positioned near to the scalp. It generates rapidly changing magnetic pulses that induce an electric field, having a modulatory effect on cortical excitability. It results in depolarization of the underlying region of the brain. Additional description of transcranial magnetic stimulation in U.S. Pat. No. 11,786,747 is incorporated by reference herein for all purposes.

In some implementations, non-invasive neurostimulation may comprise continuous transcranial magnetic stimulation (cTMS) delivery.

In some implementations, non-invasive neurostimulation may comprise transcranial direct current stimulation (tDCS) delivery. Transcranial direct-current stimulation (tDCS) is a form of non-invasive brain stimulation that shifts cortical excitability to a state of excitation of inhibition. Transcranial direct current stimulation (tDCS) passes current through the scalp, skull, and meninges in order to stimulate the brain, generally undetectably. A small electric current (1-2 mA) is delivered to the scalp by a battery-driven device connected to surface electrodes. The current is not sufficient to generate action potentials, but instead alters neuronal resting membrane potentials. Like rTMS, tDCS can alter cortical excitability.

In some implementations, non-invasive neurostimulation may comprise transcranial alternating current stimulation (tACS) delivery. tACS involves direct delivery of alternating electric currents to the scalp. The current travels through the skull to affect mostly cortical neurons. Such alternating current has a sinusoidal waveform where the voltage changes gradually from positive to negative every half-cycle. Therefore, the current flows from an anodal electrode to a cathodal electrode in one half-cycle and in the reverse direction in the second half-cycle. The concept underlying alternating current is to simulate the naturally occurring rhythmic pattern of electrophysiological activity of the brain. The typical setup of tACS involves the application of electrodes onto the scalp, whose position and size can be modified to specifically target a certain brain region, such as the LH and or the dlHPC. For this purpose, positioning of the electrodes is designed according to computational models to optimize the stimulation parameters. Furthermore, the parameters of the alternating current itself can be customized in terms of frequency, amplitude, phase shape, phase timing, and the duration and number of stimulation sessions. Additionally, the parameters may be controlled using any of the concepts described herein, such as by being programmed into a patient or clinician device.

One aspect of the disclosure, as represented in exemplary FIG. 5, is a method of treating dysregulated eating behavior in a subject, comprising: in a subject with an impaired orexigenic appetitive processing circuit mediating activity between a lateral hypothalamus (LH) and a dorsolateral hippocampus (dlHPC), invasively and/or non-invasively delivering targeted neurostimulation to at least partially repair the impaired circuit. Delivering targeted neurostimulation to at least partially repair the impaired circuit may comprise invasive DBS, an shown in FIG. 5, or non-invasive neurostimulation (or optionally some combination thereof).

In some implementations of invasive neurostimulation, delivering DBS comprises delivering continuous or near-continuous DBS targeting at least a portion of the impaired circuit, such as the LH and/or the dlHPC. Continuous DBS may refer to DBS that is not responsive to and not dependent on sensed brain electrical activity. The DBS system may have preset delivery parameters, which may be constant during the continuous DBS treatment, or which may be programmed to vary throughout one or more epochs of time during the DBS treatment. Continuous DBS may include one or more periods (temporarily ceasing) without neurostimulation.

In some implementations of invasive neurostimulation, the method may include closed-loop (e.g., intermittent) DBS targeting at least a portion of the circuit, such as a LH and/or a dlHPC. Closed loop in this context refers to DBS that is at least partially based on sensed brain activity (or one or more other sensed patient parameter), and in some particular implementations is responsive to low frequency (4-6 Hz) in the dlHPC, which is known to modulate sweet-fat cue and which is discussed in more detail in the Barbosa article incorporated by reference herein. The closed loop DBS may be programmed to initiate stimulation in response to one or more programmed sensed signals and/or events. The closed loop nature of the stimulation may optionally be adapted to be modified as needed, such as reprogramming the system by changing one or more aspects of the sensed signals that triggers the stimulation. In reference to exemplary FIGS. 1-4, sensing may occur utilizing electrodes on leads 24/42 and 26/44, by way of example only. A therapy module, an example of which is shown in FIG. 3, may initiate neurostimulation with the electrodes in response to the detected event/signal(s).

FIG. 6 illustrates a merely exemplary and illustrative method of closed loop DBS approach. The method includes sensing or monitoring brain activity signals. In response to detecting one or more signals of interest (e.g., 4-6 Hz range as described in the Barbosa reference incorporated by reference), the DBS may be initiated (e.g., automatically), and optionally ceased when the one or more signal of interest or event(s) are no longer detected.

In some implementations of invasive neurostimulation, the method and system may include scheduled (programmed) DBS targeting at least a portion of the circuit, such as the LH and/or the dlHPC. The schedule can be programmed into the system, such as into an IMD/IPG or external device if the system is a non-invasive approach. The schedule may be adapted to be modified as needed over time by reprogramming the system, such as by wirelessly reprogramming the device on which one or more computer executable methods are stored.

DBS that is programmed or scheduled to occur at certain times may include, for example only, DBS only during the day, or only at night, or only at or before mealtimes, etc.

In some implementations of invasive neurostimulation, the method and system may include patient-controlled DBS targeting the LH and/or the dlHPC. Patient control may occur through the use of, for example, a patient device such as device 134 shown in FIG. 4, that is in communication with an IMD/IPG (e.g., a smartphone with Bluetooth enabled communication).

For example only, a patient may initiate DBS at or near a time of craving. As a safety feature, the system may be adapted to sense brain activity signals in response to an indication that a patient has attempted to initiate DBS. The system may attempt to determine if sensed brain activity signals confirm that DBS should be initiated.

In some implementations, a patient can initiate neurostimulation using a patient magnet or patient controller (such as if the system is a non-invasive system).

In any of the implementations herein, the stimulation frequencies may be or comprise high frequency stimulation (>100 Hz).

In any of the implementations herein, the stimulation frequencies may be or comprise Low frequency stimulation (<4 Hz).

In any of the implementations herein, the stimulation frequencies may comprise low frequency stimulation matching the low frequency EEG signal power described in the referenced publication incorporated by reference herein (i.e., 4-6 Hz).

Even if not specifically indicated, one or more methods or techniques described in this disclosure (e.g. any of the computer executable methods) may be implemented, at least in part, in hardware, software, firmware or any combination thereof. For example, various aspects of the techniques or components may be implemented within one or more processors, including one or more microprocessors, digital signal processors (DSPs), application specific integrated circuits (ASICs), field programmable gate arrays (FPGAs), programmable logic circuitry, or the like, either alone or in any suitable combination. The term “processor” or “processing circuitry” may generally refer to any of the foregoing circuitry, alone or in combination with other circuitry, or any other equivalent circuitry.

Such hardware, software, or firmware may be implemented within one device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read only memory (ROM), non-volatile RAM (NVRAM), electrically erasable programmable ROM (EEPROM), Flash memory, and the like. The instructions may be executed by a processor to support one or more aspects of the functionality described in this disclosure.

1. A method of treating dysregulated eating behavior in a subject, comprising:
in a subject with an impaired orexigenic appetitive processing circuit mediating activity between a lateral hypothalamus (LH) and a dorsolateral hippocampus (dlHPC), invasively and/or non-invasively delivering targeted neurostimulation to at least a portion of the impaired circuit to at least partially repair the impaired circuit.

2. The method of claim 1, wherein invasively and/or non-invasively delivering neurostimulation to at least a portion of the impaired circuit comprises invasively and/or non-invasively delivering targeted neurostimulation to at least one of the LH or the dlHPC.

3. The method of claim 1, comprising invasively delivering targeted neurostimulation to at least partially repair the impaired circuit.

4. The method of claim 3, wherein invasively delivering targeted neurostimulation comprises invasively delivering neurostimulation to at least one of the LH or the dlHPC to at least partially repair the impaired circuit.

5. The method of claim 4, wherein invasively delivering neurostimulation comprises continuous deep brain stimulation (“DBS”) to at least one of the LH or the dlHPC.

6. The method of claim 4, wherein invasively delivering neurostimulation comprises closed-loop DBS to at least one of the LH or the dlHPC.

7. The method of claim 6, wherein the closed-loop DBS in responsive to low frequency (4-6 Hz) in the dlHPC known to modulate sweet-fat cue.

8. The method of claim 4, wherein invasively delivering neurostimulation comprises scheduled DBS to at least one of the LH or the dlHPC.

9. The method of claim 8, wherein the scheduled DBS occurs during at least one of only during the day, only during the night, or only at mealtimes.

10. The method of claim 4, wherein invasively delivering neurostimulation comprises at least partially patient-controlled DBS to at least one of the LH or the dlHPC, optionally wherein patient initiates stimulation using an external patient controller and/or a patient magnet.

11. The method of claim 10, wherein the patient-controlled DBS occurs at or near times of craving.

12. The method of claim 3, wherein invasively delivering neurostimulation comprises continuous or duty-cycle delivery of stimulation to a vagus nerve.

13. The method of claim 3, wherein invasively delivering neurostimulation comprises patient-controlled delivery of stimulation to a vagus nerve.

14. The method of claim 1, comprising non-invasively delivering targeted brain neurostimulation to at least partially repair the impaired circuit.

15. The method of claim 14, wherein non-invasively delivering targeted brain neurostimulation comprises repetitive transcranial magnetic stimulation (rTMS) delivery.

16. The method of claim 14, wherein non-invasively delivering targeted brain neurostimulation comprises continuous transcranial magnetic stimulation (cTMS) delivery.

17. The method of claim 14, wherein non-invasively delivering targeted brain neurostimulation comprises transcranial direct current stimulation (tDCS) delivery.

18. The method of claim 14, wherein non-invasively delivering targeted brain neurostimulation comprises transcranial alternating stimulation (tACS) delivery.

19. The method of claim 1, wherein invasively and/or non-invasively delivering neurostimulation comprises delivering high frequency stimulation (>100 Hz).

20. The method of claim 1, wherein invasively and/or non-invasively delivering neurostimulation comprises delivering low frequency stimulation (<4 Hz).

21. The method of claim 1, wherein invasively and/or non-invasively delivering neurostimulation comprises low frequency stimulation matching the low frequency EEG signal power described in the Barbosa article incorporated by reference (4-6 Hz).

22. The method of claim 1, wherein delivering brain neurostimulation comprises stimulating the impaired circuit.

23. A method of treating dysregulated eating behavior in a subject, comprising:
in a subject with an impaired orexigenic appetitive processing circuit mediating activity between a lateral hypothalamus (LH) and a dorsolateral hippocampus (dlHPC), invasively and/or non-invasively delivering brain neurostimulation to at least one of the LH or dlHPC to at least partially repair the impaired circuit.

24. A system comprising:
one or more processors; and
one or more storage media coupled to the one or more processors and storing instructions that, when executed by the one or more processors, performs a computer-implemented method comprising:
causing neurostimulation to be delivered to at least a portion of impaired orexigenic appetitive processing circuit mediating activity between a lateral hypothalamus (LH) and a dorsolateral hippocampus (dlHPC).

25. The system of claim 24, wherein the one or more processors and the one or more storage media are disposed in an implantable medical device.

26. The system of claim 24, wherein the storing instructions that, when executed by the one more processors, performs a computer-implemented method comprising causing continuous DBS to be delivered to at least a portion of the impaired circuit.

27. The system of claim 24, wherein the storing instructions that, when executed by the one more processors, performs a computer-implemented method comprising closed-loop DBS to least a portion of the impaired circuit.

28. The system of claim 27, wherein the closed-loop DBS is responsive to low frequency (4-6 Hz) in the dlHPC known to modulate sweet-fat cue.

29. The system of claim 24, wherein the storing instructions that, when executed by the one or more processors, performs a computer-implemented method comprising scheduled DBS to at least a portion of the impaired circuit.

30. The system of claim 29, wherein the storing instructions are adapted to schedule DBS to occur during at least one of only during the day, only during the night, or only at mealtimes.

31. The system of claim 24, wherein the storing instructions that, when executed by the one or more processors, performs a computer-implemented method comprising at least partially patient-controlled DBS to at least a portion of the impaired circuit.

32. The system of claim 31, wherein the system comprises an implantable pulse generator, and wherein the implantable pulse generator is adapted to be communication with a patient controller, the patient controller, in response to user actuation of the patient controller, facilitating the initiating of the DBS.

33. The system of claim 24, wherein the system is a non-invasive neurostimulation system, optionally wherein the system includes a patient controller (such as any of those described herein) that is adapted to initiate the non-invasive neurostimulation.

34. The system of claim 33, wherein the system comprises a repetitive transcranial magnetic stimulation device.

35. The system of claim 33, wherein the system comprises a continuous transcranial magnetic stimulation device.

36. The system of claim 33, wherein the system comprises a transcranial direct current stimulation device.

37. The system of claim 33, wherein the system comprises a transcranial alternating stimulation device.

38. The system of claim 24, wherein causing neurostimulation to be delivered to at least a portion of impaired circuit, optionally initiated by a patient with a patient controller, comprises delivering high frequency stimulation (>100 Hz) to at least a portion of the impaired circuit.

39. The system of claim 24, wherein causing neurostimulation to be delivered to at least a portion of impaired circuit, optionally initiated by a patient with a patient controller, comprises delivering low frequency stimulation (<4 Hz) to at least a portion of the circuit.

40. The system of claim 24, wherein causing neurostimulation to be delivered to at least a portion of impaired circuit, optionally initiated by a patient with a patient controller, comprises low frequency stimulation matching the low frequency EEG signal power (4-6 Hz) described in the Barbosa article incorporated by reference.

41. The system of claim 24, further comprising one or more brain leads, each with one or more electrodes thereon.
